SEQUENCE LISTING (28 sequences)

SEQ ID NO: 1 - protein, 384 aa, Homo sapiens
MAAQNGNTSFTPNFNPPQDHASSLSFNFSYGDYDLPMDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPTTEEVDCIRLK

SEQ ID NO: 2 - protein, 456 aa, Homo Sapiens
LWGRSANSSWNPETLTRRRSPPGRGKKRDIPHAAALQVQVRVLEVKTERPACAQPHCDSRSGFRSSRAHRKEAPRQNGNTSFTPNFNPPQDHASSLSFNFSYGDYDLPMDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPTTEEVDCIRLK

SEQ ID NO: 3 - protein, 442 aa, Homo Sapiens
LTRRRSPPGRGKKRDIPHAAALQVQVRVLEVKTERPACAQPHCDSRSGFRSSRAHRKEAPRQNGNTSFTPNFNPPQDHASSLSFNFSYGDYDLPMDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPTTEEVDCIRLK

SEQ ID NO: 4 - protein, 421 aa, Homo sapiens
LQVQVRVLEVKTERPACAQPHCDSRSGFRSSRAHRKEAPRQNGNTSFTPNFNPPQDHASSLSFNFSYGDYDLPMDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPTTEEVDCIRLK

SEQ ID NO: 5 - protein, 347 aa, Homo sapiens
MDEDEDMTTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPTTEEVDCIRLK

SEQ ID NO: 6 - protein, 386 aa, Homo sapiens
MDDNATNTSTSFLSVLNPHGAHATSFPFNFSYSDYDMPLDEDEDVTNSRTFFAAKIVIGMALVGIMLVCGIGNFIFIAALVRYKKLRNLTNLLIANLAISDFLVAIVCCPFEMDYYVVRQLSWEHGHVLCTSVNYLRTVSLYVSTNALLAIAIDRYLAIVHPLRPRMKCQTATGLIALVWTVSILIAIPSAYFTTETVLVIVKSQEKIFCGQIWPVDQQLYYKSYFLFIFGIEFVGPVVTMTLCYARISRELWFKAVPGFQTEQIRKRLRCRRKTVLVLMCILTAYVLCWAPFYGFTIVRDFFPTVFVKEKHYLTAFYIVECIAMSNSMINTLCFVTVKNDTVKYFKKIMLLHWKASYNGGKSSADLDLKTIGMPATEEVDCIRLK

SEQ ID NO: 7 - protein, 393 aa, Homo sapiens
METTMGFMDDNATNTSTSFLSVLNPHGAHATSFPFNFSYSDYDMPLDEDEDVTNSRTFFAAKIVIGMALVGIMLVCGIGNFIFIAALVRYKKLRNLTNLLIANLAISDFLVAIVCCPFEMDYYVVRQLSWEHGHVLCTSVNYLRTVSLYVSTNALLAIAIDRYLAIVHPLRPRMKCQTATGLIALVWTVSILIAIPSAYFTTETVLVIVKSQEKIFCGQIWPVDQQLYYKSYFLFIFGIEFVGPVVTMTLCYARISRELWFKAVPGFQTEQIRKRLRCRRKTVLVLMCILTAYVLCWAPFYGFTIVRDFFPTVFVKEKHYLTAFYIVECIAMSNSMINTLCFVTVKNDTVKYFKKIMLLHWKASYNGGKSSADLDLKTIGMPATEEVDCIRLK

SEQ ID NO: 8 - DNA, 1316 bp, Homo sapiens
aagtgaagggctccagactctccggacagatgggctgaggcactcctgggattgtctttatacggccatggaatctgatagtggtggtcatcgcaaagtttgggtgaggacttcaggggctttgagcagagcaaactgtccccatccccagcttcctcctgggggcctcctccttcaccgccatctccaaaggcccacaccgcactcgtcggagccgggctttccttgaaagggtcaccttctagtgggcgcggacgctggcactgggcacctccggaagacaggactctcccctcctgcgcgcgcgacccgggtgcggtgccacgactggcggcagagggcggtgctcactgttcccaagcgcacggtagaaacccggctactgtgtggtccgattcagacgggatgcgaaggcgctgaaatccgtgcgagaggctgcccccgctgccggggactcgcgtcagcgcggccggagctacggtgcccgcgccctggacgcaccagcccgagagcacgcggtggggtgggggagctgggcatccacccttggcctggactaggaggcaggcggggaccccggggcggagtccagcgcggggcggggcgcacaggtccgagacgctggaaagagctgtggggccggagcgccaactccagctggaacccagagaccctgacacgccggaggagcccgccggggcgaggaaaaaagagggacatcccccacgcagcggctctgcaagtgcaagtccgggtcctcgaggtgaaaaccgagcggcccgcctgcgcccaaccacactgtgactctcgatccggcttccggagcagcagagcgcacagaaaggaggcgccgagacagaatggaaacaccagtttcacacccaactttaatccaccccaagaccatgcctcctccctctcctttaacttcagttatggtgattatgacctccctatggatgaggatgaggacatgaccaagacccggaccttcttcgcagccaagatcgtcattggcattgcactggcaggcatcatgctggtctgcggcatcggtaactttgtctttatcgctgccctcacccgctataagaagttgcgcaacctcaccaatctgctcattgccaacctggccatctccgacttcctggtggccatcatctgctgccccttcgagatggactactacgtggtacggcagctctcctgggagcatggccacgtgctctgtgcctccgtcaactacctgcgcaccgtctccctctacgtctccaccaatgccttgctggccattgccattgacaggtgaggatggt

SEQ ID NO: 9 - DNA, 1336 bp, Homo sapiens
aagtgaagggctccagactctccggacagatgggctgaggcactcctgggattgtctttatacggccatggaatctgatagtggtggtcatcgcaaagtttgggtgaggacttcaggggctttgagcagagcaaactgtccccatccccagcttcctcctgggggcctcctccttcaccgccatctccaaaggcccacaccgcactcgtcggagccgggctttccttgaaagggtcaccttctagtgggcgcggacgctggcactgggcacctccggaagacaggactctcccctcctgcgcgcgcgacccgggtgcggtgccacgactggcggcagagggcggtgctcactgttcccaagcgcacggtagaaacccggctactgtgtggtccgattcagacgggatgcgaaggcgctgaaatccgtgcgagaggctgcccccgctgccggggactcgcgtcagcgcggccggagctacggtgcccgcgccctggacgcaccagcccgagagcacgcggtggggtgggggagctgggcatccacccttggcctggactaggaggcaggcggggaccccggggcggagtccagcgcggggcggggcgcacaggtccgagacgctggaaagagctgtggggccggagcgccaactccagctggaacccagagaccctgacacgccggaggagcccgccggggcgaggaaaaaagagggacatcccccacgcagcggctctgcaagtgcaagtccgggtcctcgaggtgaaaaccgagcggcccgcctgcgcccaaccacactgtgactctcgatccggcttccggagcagcagagcgcacagaaaggaggcgccgagacagacatcaccatggcagcccagaatggaaacaccagtttcacacccaactttaatccaccccaagaccatgcctcctccctctcctttaacttcagttatggtgattatgacctccctatggatgaggatgaggacatgaccaagacccggaccttcttcgcagccaagatcgtcattggcattgcactggcaggcatcatgctggtctgcggcatcggtaactttgtctttatcgctgccctcacccgctataagaagttgcgcaacctcaccaatctgctcattgccaacctggccatctccgacttcctggtggccatcatctgctgccccttcgagatggactactacgtggtacggcagctctcctgggagcatggccacgtgctctgtgcctccgtcaactacctgcgcaccgtctccctctacgtctccaccaatgccttgctggccattgccattgacaggtgaggatggt

SEQ ID NO: 10 - DNA, 1182 bp, Homo sapiens
atggagaccaccatggggttcatggatgacaatgccaccaacacttccaccagcttcctttctgtgctcaaccctcatggagcccatgccacttccttcccattcaacttcagctacagcgactatgatatgcctttggatgaagatgaggatgtgaccaattccaggacgttctttgctgccaagattgtcattgggatggccctggtgggcatcatgctggtctgcggcattggaaacttcatctttatcgctgccctggtccgctacaagaaactgcgcaacctcaccaacctgctcatcgccaacctggccatctctgacttcctggtggccattgtctgctgcccctttgagatggactactatgtggtgcgccagctctcctgggagcacggccacgtcctgtgcacctctgtcaactacctgcgcactgtctctctctatgtctccaccaatgccctgctggccatcgccattgacaggtatctggctattgtccatccgctgagaccacggatgaagtgccaaacagccactggcctgattgccttggtgtggacggtgtccatcctgatcgccatcccttccgcctacttcaccaccgagacggtcctcgtcattgtcaagagccaggaaaagatcttctgcggccagatctggcctgtggaccagcagctctactacaagtcctacttcctctttatctttggcatagaattcgtgggccccgtggtcaccatgaccctgtgctatgccaggatctcccgggagctctggttcaaggcggtccctggattccagacagagcagatccgcaagaggctgcgctgccgcaggaagacggtcctggtgctcatgtgcatcctcaccgcctacgtgctatgctgggcgcccttctacggcttcaccatcgtgcgcgacttcttccccaccgtgtttgtgaaggagaagcactacctcactgccttctacatcgtcgagtgcatcgccatgagcaacagcatgatcaacactctgtgcttcgtgaccgtcaagaacgacaccgtcaagtacttcaaaaagatcatgttgctccactggaaggcttcttacaatggcggtaagtccagtgcagacctggacctcaagacaattgggatgcctgccaccgaagaggtggactgcatcagactaaaataa

SEQ ID NO: 11 - DNA, 39 bp, Artificial Sequence
  primer
gcgaagcttcttgcgaatctgctccgtctggaacccagg

SEQ ID NO: 12 - DNA, 42 bp, Artificial Sequence
  primer
ctgaagcttctggaacccagggactgccttgaaccagagctc

SEQ ID NO: 13 - DNA, 27 bp, Artificial Sequence
  primer
ccatcctaatacgactcactatagggc

SEQ ID NO: 14 - DNA, 23 bp, Artificial Sequence
  primer
actcactatagggctcgagcggc

SEQ ID NO: 15 - DNA, 1273 bp, Homo sapiens
gaattcgcccttactcactatagggctcgagcggccgcccgggcaggtctcactgttcccaagcgcacggtagaaacccggctactgtgtggtccgattcagacgggatgcgaaggcgctgaaatccgtgcgagaggctgcccccgctgccggggactcgcgtcagcgcggccggagctacggtgcccgcgccctggacgcaccagcccgagagcacgcggtggggtgggggagctgggcatccacccttggcctggactaggaggcaggcggggaccccggggcggagtccagcgcggggcggggcgcacaggtccgagacgctggaaagagctgtggggccggagcgccaactccagctggaacccagagaccctgacacgccggaggagcccgccggggcgaggaaaaaagagggacatcccccacgcagcggctctgcaagtgcaagtccgggtcctcgaggtgaaaaccgagcggcccgcctgcgcccaaccacactgtgactctcgatccggcttccggagcagcagagcgcacagaaaggaggcgccgagacagttatggtgattatgacctccctatggatgaggatgaggacatgaccaagacccggaccttcttcgcagccaagatcgtcattggcattgcactggcaggcatcatgctggtctgcggcatcggtaactttgtctttatcgctgccctcacccgctataagaagttgcgcaacctcaccaatctgctcattgccaacctggccatctccgacttcctggtggccatcatctgctgccccttcgagatggactactacgtggtacggcagctctcctgggagcatggccacgtgctctgtgcctccgtcaactacctgcgcaccgtctccctctacgtctccaccaatgccttgctggccattgccattgacagatatctcgccatcgttcaccccttgaaaccacggatgaattatcaaacggcctccttcctgatcgccttggtctggatggtgtccattctcattgccatcccatcggcttactttgcaacagaaacggtcctctttattgtcaagagccaggagaagatcttctgtggccagatctggcctgtggatcagcagctctactacaagtcctacttcctcttcatctttggtgtcgagttcgtgggccctgtggtcaccatgaccctgtgctatgccaggatctcccgggagctctggttcaaggcagtccctgggttccagaagcttcagaagggcgaattc

SEQ ID NO: 16 - DNA, 1595 bp, Homo sapiens
cgcccgggcaggtctcactgttcccaagcgcacggtagaaacccggctactgtgtggtccgattcagacgggatgcgaaggcgctgaaatccgtgcgagaggctgcccccgctgccggggactcgcgtcagcgcggccggagctacggtgcccgcgccctggacgcaccagcccgagagcacgcggtggggtgggggagctgggcatccacccttggcctggactaggaggcaggcggggaccccggggcggagtccagcgcggggcggggcgcacaggtccgagacgctggaaagagctgtggggccggagcgccaactccagctggaacccagagaccctgacacgccggaggagcccgccggggcgaggaaaaaagagggacatcccccacgcagcggctctgcaagtgcaagtccgggtcctcgaggtgaaaaccgagcggcccgcctgcgcccaaccacactgtgactctcgatccggcttccggagcagcagagcgcacagaaaggaggcgccgagacagttatggtgattatgacctccctatggatgaggatgaggacatgaccaagacccggaccttcttcgcagccaagatcgtcattggcattgcactggcaggcatcatgctggtctgcggcatcggtaactttgtctttatcgctgccctcacccgctataagaagttgcgcaacctcaccaatctgctcattgccaacctggccatctccgacttcctggtggccatcatctgctgccccttcgagatggactactacgtggtacggcagctctcctgggagcatggccacgtgctctgtgcctccgtcaactacctgcgcaccgtctccctctacgtctccaccaatgccttgctggccattgccattgacagatatctcgccatcgttcaccccttgaaaccacggatgaattatcaaacggcctccttcctgatcgccttggtctggatggtgtccattctcattgccatcccatcggcttactttgcaacagaaaccgtcctctttattgtcaagagccaggagaagatcttctgtggccagatctggcctgtggatcagcagctctactacaagtcctacttcctcttcatctttggtgtcgagttcgtgggccctgtggtcaccatgaccctgtgctatgccaggatctcccgggagctctggttcaaggcagtccctgggttccagacggagcagattcgcaagcggctgcgctgccgcaggaagacggtcctggtgctcatgtgcattctcacggcctatgtgctgtgctgggcacccttctacggtttcaccatcgttcgtgacttcttccccactgtgttcgtgaaggaaaagcactacctcactgccttctacgtggtcgagtgcatcgccatgagcaacagcatgatcaacaccgtgtgcttcgtgacggtcaagaacaacaccatgaagtacttcaagaagatgatgctgctgcactggcgtccctcccagcgggggagcaagtccagtgctgaccttgacctcagaaccaacggggtgcccaccacagaagaagtggactgtatcaggctgaagtga

SEQ ID NO: 17 - protein, 348 aa, Homo sapiens
MDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPTTEEVDCIRLK

SEQ ID NO: 18 - protein, 81 aa, Homo sapiens
AVITGACDKDSQCGGGMCCAVSIWVKSIRICTPMGKLGDSCHPLTRKVPFFGRRMHHTCPCLPGLACLRTSFNRFICLAQK

SEQ ID NO: 19 - protein, 100 aa, Homo sapiens
AVITGACDKDSQCGGGMCCAVSIWVKSIRICTPMLGDSCHPLTRKNNFGNGRQERRKRKRSKRKKEVPFFGRRMHHTCPCLPGLACLRTSFNRFICLAQK

SEQ ID NO: 20 - protein, 81 aa, Mus musculus
AVITGACDKDSQCGGGMCCAVSIWVKSIRICTPMGQVGDSCHPLTRKVPFWGRRMHHTCPCLPGLACLRTSFNRFICLARK

SEQ ID NO: 21 - protein, 102 aa, Mus musculus
AVITGACDKDSQCGGGMCCAVSIWVKSIRICTPMGQVGDSCHPLTRKSHVANGRQERRRAKRRKRKKEVPFWGRRMHHTCPCLPGLACLRTSFNRFICLARK

SEQ ID NO: 22 - protein, 86 aa, Homo sapiens
AVITGACERDVQCGAGTCCAISLWLRGLRMCTPLGREGEECHPGSHKVPFFRKRKHHTCPCLPNLLCSRFPDGRYRCSMDLKNINF

SEQ ID NO: 23 - protein, 86 aa, Mus musculus
AVITGACERDIQCGAGTCCAISLWLRGLRLCTPLGREGEECHPGSHKIPFLRKRQHHTCPCSPSLLCSRFPDGRYRCFRDLKNANF

SEQ ID NO: 24 - protein, 77 aa, Xenopus laevis
AVITGACDKDVQCGSGTCCAASAWSRNIRFCIPLGNSGEDCHPASHKVPYDGKRLSSLCPCKSGLTCSKSGEKFKCS

SEQ ID NO: 25 - protein, 75 aa, Bufo sp.
AVITGACDKDVQCGSGTCCAASAWSRNIRFCIPLSGEDCHPASHKVPYDGKRLSSLCPCKSGLTCSKSGEKFKCS

SEQ ID NO: 26 - protein, 80 aa, Serpentes sp.
AVITGACERDLQCGKGTCCAVSLWIKSVRVCTPVGTSGEDCHPASHKIPFSGQRMHHTCPCAPNLACVQTSPKKFKCLSK

SEQ ID NO: 27 - protein, 81 aa, Artificial Sequence
  Chimera PK1/PK2
AVITGACERDVQCGAGTCCAISLWLRGLRMCTPLGREGEECHPGSHKVPFFGRRMHHTCPCLPGLACLRTSFNRFICLAQK

SEQ ID NO: 28 - protein, 86 aa, Artificial Sequence
  Chimera PK2/PK1
AVITGACDKDSQCGGGMCCAVSIWVKSIRICTPMGKLGDSCHPLTRKVPFFRKRKHHTCPCLPNLLCSRFPDGRYRCSMDLKNINF

(19) United States
(12) Patent Application Publication
(10) Pub. No.: US 2005/0170455 A1
(43) Pub. Date: Aug. 4, 2005

(54) Novel prokineticin receptor isoforms and methods of use

(76) Inventor: Qun-Yong Zhou, Irvine, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 10/871,152

(22) Filed: Jun. 18, 2004

Related U.S. Application Data
(60) Provisional application No. 60/480,239, filed on Jun. 20, 2003.

(51) Int. Cl. C07K 14/705; C07K 16/28; C07H 21/04

(52) U.S. Cl. 435/69.1; 435/320.1; 435/325; 530/350; 530/388.22; 536/23.5

(57) ABSTRACT

The invention provides an isolated prokineticin receptor 2 long isoform polypeptide that contains an amino acid sequence selected from the amino acid sequences referenced as SEQ ID NO:2, SEQ ID NO:3, or SEQ ID NO:4. Also provided is an isolated prokineticin receptor 2 short isoform polypeptide, which contains the amino acid sequence referenced as SEQ ID NO:5. Also provided is further prokineticin 2 short isoform polypeptide containing the sequence of SEQ ID NO:17. Further provided is an isolated prokineticin receptor 1 short isoform polypeptide that contains the amino acid sequence referenced as SEQ ID NO:6. The invention also provides methods for preparing an isolated polypeptide corresponding to a long or short PKR isoform of the invention; as well as antibodies That selectively bind to a long or short PKR isoform of the invention. Methods for identifying agonists and antagonists of PKR1 and PKR2 further are provided by the invention.

This application claims benefit of the filing date of U.S. Provisional Application No. 60/480,239, filed Jun. 20, 2003, and which is incorporated herein by reference.

### BACKGROUND OF THE INVENTION

This invention relates generally to the field of medicine, and more specifically, to newly identified isoforms of prokineticin receptors useful for drug discovery and diagnostic testing.

Proteins are major building blocks of cells that serve many vital functions both within cells and as extracellular molecules. Thousands of different proteins are present in each cell of our bodies, with the synthesis of each protein being directed by a specific gene. Although some genes encode a single protein, it is recognized that in many cases, one gene can encode multiple protein forms, referred to as isoforms. One way that variant protein isoforms arise is through alternative RNA splicing.

RNA splicing is the process that takes place in eukaryotic nuclei in which introns, or non-coding RNA sequences, are removed from primary RNA transcripts prior to the ligation of exons, or coding RNA sequences, to form functional messenger RNA. By altering how the “pre-mRNA” is spliced, for example, to include or exclude one or more “optional” exons, different versions of the mRNA can be produced. Each different version of mRNA can then be translated in the cell to produce a different protein isoform.

Alternative splicing is a widely occurring phenomenon, with at least 30% of human genes exhibiting alternative splicing. Some pre-mRNAs are alternatively spliced in different cell types or at different times during development, giving rise to different cell- or tissue-specific isoforms or developmentally-restricted isoforms. Furthermore, these and other splice variant forms can encode protein isoforms that have physiological activities that differ in degree or type from related isoforms. An isoform arising from a splice variant form can differ, for example, in stability, clearance rate, tissue or cellular localization, tissue expression pattern, temporal pattern of expression, regulation, or response to agonists or antagonists.

In many cases, the presence or level of a specific isoform contributes to, or protects against, a pathological condition. As such, some protein isoforms represent new drug targets or diagnostic markers. Because a drug can have differential activity on one isoform compared to another, knowledge of isoforms that represent drug targets can contribute to improved understanding of drug effectiveness, as well as improved drug screening strategies and drug design.

Prokineticin receptors are G-protein coupled receptors important in several biological functions, including circadian rhythm function; angiogenesis; gastric contractility and motility; gastric acid and pepsinogen secretion; pain; and neurogenesis. Different prokineticin receptor isoforms can have roles in particular tissues or conditions associated with altered prokineticin receptor function. Newly identified isoforms of prokineticin receptors can therefore serve as drug targets or diagnostic markers.

Thus, there exists a need for identifying alternative isoforms of prokineticin receptors. The present invention satisfies this need and provides related advantages as well.

### SUMMARY OF THE INVENTION

The invention provides short and long isoforms of two forms of prokineticin receptor (PKR). The invention provides an isolated prokineticin receptor 2 long isoform polypeptide that contains an amino acid sequence selected from the amino acid sequences referenced as SEQ ID NO:2, SEQ ID NO:3, or SEQ ID NO:4. Also provided is an isolated prokineticin receptor 2 short isoform polypeptide, which contains the amino acid sequence referenced as SEQ ID NO:5. The invention further provides an isolated prokineticin receptor 2 (PKR2) short isoform polypeptide that contains the amino acid sequence referenced as SEQ ID NO:17. The invention further provides an isolated prokineticin receptor 1 (PKR1) short isoform polypeptide that contains the amino acid sequence referenced as SEQ ID NO:6.

The invention provides methods for preparing an isolated polypeptide corresponding to a long or short PKR isoforms of the invention. The method involves culturing a host cell that expresses the polypeptide, and substantially purifying the polypeptide. Also provided are antibodies that selectively bind to a long or short PKR isoform of the invention.

The invention provides a method of identifying a prokineticin 2 receptor agonist. The method involves contacting a preparation comprising a prokineticin 2 receptor isoform polypeptide selected from SEQ ID NO:2, SEQ ID NO:3, SEQ ID NO:4, SEQ ID NO:5 or SEQ ID NO:17, with one or more candidate compounds, and identifying a compound that selectively promotes production of a prokineticin 2 receptor signal, the compound being characterized as an agonist of said prokineticin 2 receptor isoform.

Also provided is a method of identifying a prokineticin 2 receptor antagonist. The method involves contacting a preparation comprising a prokineticin 2 receptor polypeptide isoform selected from SEQ ID NO:2, SEQ ID NO:3, SEQ ID NO:4, SEQ ID NO:5 or SEQ ID NO:17 with one or more candidate compounds in the presence of a prokineticin, and identifying a compound that selectively inhibits production of a prokineticin 2 receptor signal, the compound being characterized as an antagonist of said prokineticin 2 receptor isoform.

Further provided is a method of identifying a prokineticin 1 receptor agonist. The method involves contacting a preparation comprising a prokineticin 1 receptor polypeptide isoform referenced as SEQ ID NO:6, with one or more candidate compounds, and identifying a compound that selectively promotes production of a prokineticin 1 receptor signal, the compound being characterized as an agonist of said prokineticin 1 receptor isoform.

Also provided is a method of identifying a prokineticin 1 receptor antagonist. The method involves contacting a preparation comprising a prokineticin 1 receptor polypeptide isoform referenced as SEQ ID NO:6, with one or more candidate compounds in the presence of a prokineticin, and identifying a compound that selectively inhibits production of a prokineticin 1 receptor signal, the compound being characterized as an antagonist of the prokineticin 1 receptor isoform.

### BRIEF DESCRIPTION OF THE DRAWINGS

FIG. 1 shows the amino acid sequences of (A) human prokineticin receptor 2 (PKR2) (SEQ ID NO:1); (B) PKR2 long isoform 632 encoded by nucleotide sequence SEQ ID NO:8, beginning at nucleotide 632(SEQ ID NO:2); (C) PKR2 long isoform encoded by nucleotide sequence SEQ ID NO:8, beginning at nucleotide 674 (SEQ ID NO:3); (D) PKR2 long isoform encoded by nucleotide sequence SEQ ID NO:8, beginning at nucleotide 737 (SEQ ID NO:4); (E) PKR2 short isoform encoded by nucleotide sequence SEQ ID NO:8, beginning at nucleotide 966 (SEQ ID NO:5); (F) originally recognized human prokineticin receptor 1 (PKR1) (SEQ ID NO:7); (G) PKR1 short isoform encoded by nucleotide sequence SEQ ID NO:10, beginning at nucleotide 25 (SEQ ID NO:6); (H) variant isoform PKR2 nucleotide sequence (SEQ ID NO:8); (I) originally recognized PKR2 nucleotide sequence (SEQ ID NO:9) and (J) PKR1 nucleotide sequence (SEQ ID NO:10).

FIG. 3A shows the nucleotide sequence of one PKR2 5′ RACE primer (SEQ ID NO:11); FIG. 3B, of another PKR2 5′ RACE primer (3B: SEQ ID NO:12); FIG. 3C, of a RACE adaptor primer, AP1 (SEQ ID NO:13); and FIG. 3D, of another RACE adaptor primer, AP2 (SEQ ID NO:14).

FIG. 4 (SEQ ID NO:15) shows a RACE PCR product using PKR2 5′ RACE primers R1 and R2; and 3′ RACE primers AP1 and AP2.

FIG. 5 (SEQ ID NO:16) shows a nucleotide sequence of an isoform of PKR2.

FIG. 6 shows an amino acid sequence of a short isoform of human PKR2 (SEQ ID NO:17).

### DETAILED DESCRIPTION OF THE INVENTION

This invention is directed to newly identified isoforms of prokineticin (PK) receptors, nucleic acids encoding the PK receptor isoforms, and to methods for using the PK receptor isoforms, for example, in identifying compounds that modulate PK receptor activity.

As described herein, the identification by the inventor of intronic sequence in the 5′ untranslated region of the human PK2 receptor (PKR2) gene sequence contributed to the identification of previously unrecognized isoforms of PKR2. The short and long PK receptor isoforms of the invention differ at their N-termini from the originally recognized isoforms of PK receptors. The invention short PK2 receptor isoform contains an N-terminal deletion with respect to the human PK2 receptor SEQ ID NO:1, whereas the long PK2 receptor isoforms contain N-terminal additions with respect to human PK2 receptor SEQ ID NO:1. Similarly, the invention short PK1 receptor isoform contains an N-terminal deletion with respect to human PK1 receptor SEQ ID NO:7.

Comparison of human PKR2 cDNA sequence and genomic sequence revealed the presence of at least three exons for human PKR2 gene. Thus, it was determined that isoforms of PKR2 can arise from the phenomenon of alternative splicing. Particularly, the isoforms of PKR2 (SEQ ID NOS: 2, 3 and 4) can be produced using an alternative splicing acceptor site that is 20 bp downstream of a canonical acceptor site. Utilizing the canonical acceptor site produces PKR2 referenced as SEQ ID NO:1. The PKR2 isoform referenced as SEQ ID NO:5 arises from utilization of a downstream starting ATG from a mRNA that utilizes the canonical or alternative splicing acceptor site, and thus produces a receptor protein 36 residues shorter. The PKR1 isoform referenced as SEQ ID NO:6 arises from utilizing of a downstream starting ATG that will produce a receptor protein 7 residues shorter than that originally characterized (SEQ ID NO:7). The short PKR2 isoform of SEQ ID NO:17 that is expressed in both human hypothalamus and thalamus, which are crucial circadian clock targets.

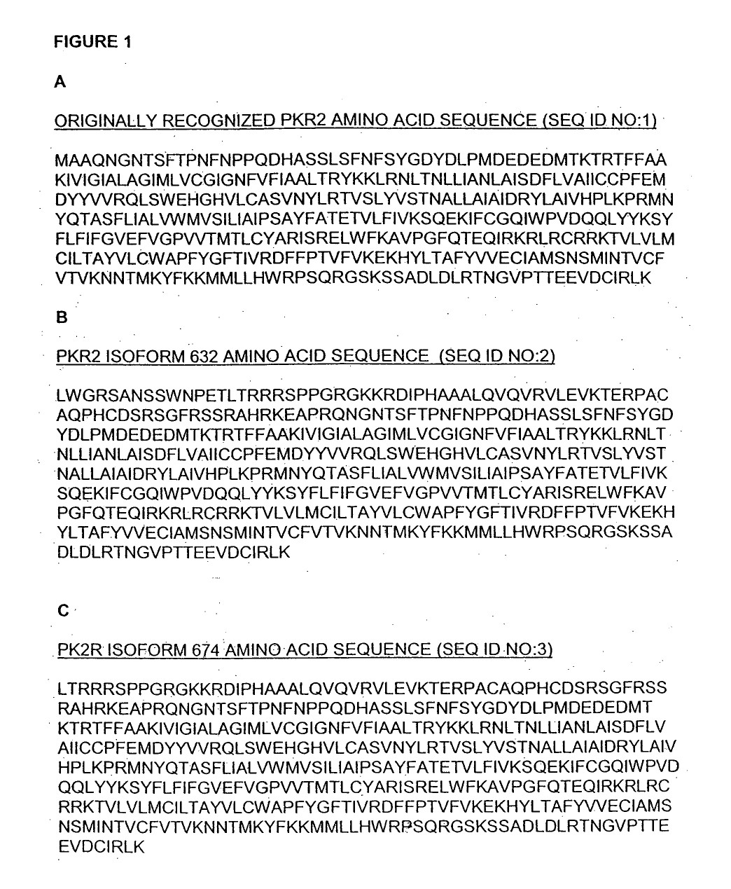

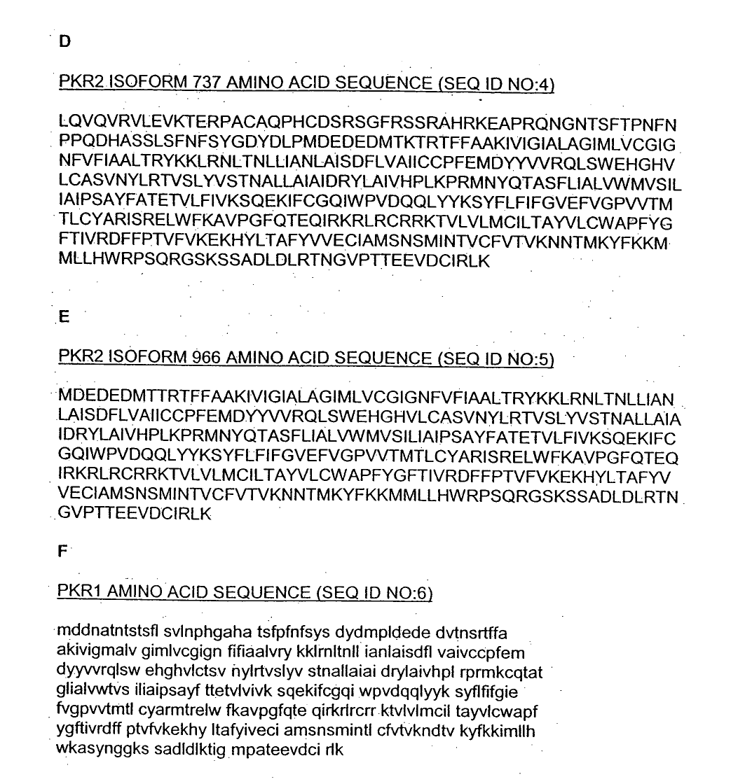

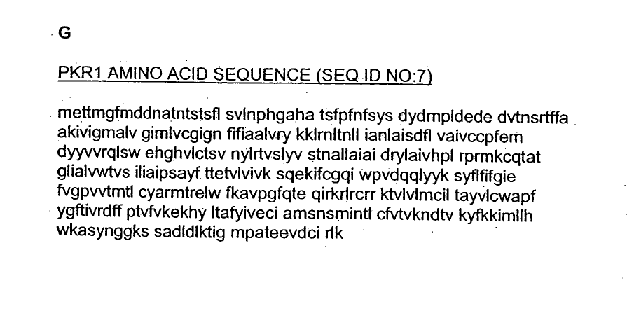

The originally recognized human PKR2 is encoded by the nucleotide sequence shown in FIG. 1 (SEQ ID NO:9), with the first coding nucleotide being nucleotide 867 of this sequence. The identified long isoforms of PKR2 are encoded by the variant PKR2 nucleotide sequence shown in FIG. 1 (SEQ ID NO:8), with first coding nucleotide being at either nucleotide 632; nucleotide 674; or nucleotide 737 of this sequence. A short isoform of PKR2 is encoded by the nucleotide sequence shown in FIG. 1 (SEQ ID NO:8), with first coding nucleotide being nucleotide 966.

Therefore, the invention provides an isolated prokineticin receptor 2 long isoform polypeptide, containing an amino acid sequence selected from the amino acid sequences referenced as: SEQ ID NO:2, SEQ ID NO:3, or SEQ ID NO:4. Polypeptides having amino acid sequences of the long and short PKR2 isoforms are referenced as follows:

TABLE 1
PRK2 isoforms

| PKR2 isoform amino | First nucleotide of |
|---|---|
| acid sequence | variant human PKR2 (SEQ ID |
| SEQ ID NO | NO: 8) encoding the isoform |
| SEQ ID NO: 2 | 632 |
| SEQ ID NO: 3 | 674 |
| SEQ ID NO: 4 | 737 |
| SEQ ID NO: 5 | 966 |

The invention also provides a short isoform of prokineticin 1 receptor (PKR1). The amino acid sequence of the short isoform of PKR1 is referenced as SEQ ID NO:6, and is encoded by a nucleotide sequence beginning at nucleotide 25 of human PKR1 SEQ ID NO:10.

The invention also provides a short isoform of PKR2 (SEQ ID NO:17), which is expressed in the hypothalamus and thalamus. This short isoform is encoded by the sequence shown in SEQ ID NO:16.

The invention further provides methods for expressing the identified long and short PK receptor isoforms, and for using them to identify PK receptor modulating compounds, such as PK receptor agonists and antagonists. Because the identified long and short PK receptor isoforms can represent different cell- or tissue-specific isoforms or isoforms that have physiological activities that differ from the originally recognized PK receptors, their presence in a cell or tissue can correlate with a disease or other unwanted condition. In addition, because a short or long PK receptor isoform can differ from the originally recognized PK receptors with respect to stability, clearance rate, tissue or cellular localization, tissue expression pattern, temporal pattern of expression, regulation, or response to agonists or antagonists, it can be useful to preferentially modulate the activity of a short or long isoform of PK receptor. Also, because the invention short or long PK receptor isoforms can have substantially the same activity as each other or as the originally recognized PK receptors, they can be used to identify compounds for modulating one or more PK receptor isoforms, or a particular isoform.

The long isoforms of PKR2 of the invention are predicted to bind to PK2 and/or PK1 and signal through a G-protein coupled signal transduction pathway in response to PK2. Similarly, the short isoform of PKR1 of the invention is predicted to bind to PK1 and/or PK2 and signal through a G-protein coupled signal transduction pathway in response to PK1. Such binding and signaling activity is predicted because structure-function studies of G-protein coupled receptors (GPCRs) indicates that GPCRs having N-terminal amino acid additions or deletions generally maintain receptor function. In contrast, small ligands generally make contact with residues in several transmembrane helices and may also make contact with residues in the extracellular domain (Flower, Biochimica et Biophysica Acta 1422: 207-234 (1999)). In addition, G-proteins generally make contact with the second intracellular loop and with the N and C segments of the third intracellular loop of the receptor (Wess, Pharmacol. Ther. 80: 231-264 (1998)).

The invention provides long and short isoforms of human PK2 receptor (PKR2). As used herein, the term “human PK2 receptor” or “PKR2” means a heptahelical membrane-spanning G-protein-coupled receptor comprising the amino acid sequence of human PK2 receptor, or a naturally-occurring or man-made minor modification thereof that binds to PK2 and signals through a G-protein coupled signal transduction pathway in response to PK2. A PK2 receptor also can bind to PK1 to induce PK2 receptor signaling. The amino acid sequence referenced as SEQ ID NO:1, which corresponds to the originally recognized human PK2 receptor isoform, is encoded by the nucleotide sequence referenced as SEQ ID NO:9. The newly identified variant human PK2 receptor isoform is encoded by the nucleotide sequence referenced as SEQ ID NO:8. The term “long isoform,” as used herein means a PK receptor polypeptide that contains additional amino acids with respect to SEQ ID NO:1 and is encoded by a PK receptor gene. As described herein, long isoforms of human PK2 receptor are referenced as SEQ ID NO:2, which is encoded by SEQ ID NO:8 beginning at nucleotide 632; SEQ ID NO:3, which is encoded by SEQ ID NO:8 beginning at nucleotide 674; and SEQ ID NO:4, which is encoded by SEQ ID NO:8 beginning at nucleotide 737. The term “short isoform,” as used herein means a PK receptor polypeptide that contains fewer amino acids with respect to SEQ ID NO:1 and is encoded by a PK receptor gene. A short isoform of human PK2 receptor is referenced as SEQ ID NO:5, which is encoded by SEQ ID NO:8 beginning at nucleotide 966.

The invention provides a short isoform of human PK1 receptor (PKR1). As used herein, the term “human PK1 receptor” or “PKR1” means a heptahelical membrane-spanning G-protein-coupled receptor comprising the amino acid sequence of human PK1 receptor, or a naturally-occurring or man-made minor modification thereof that binds to PK1 and signals through a G-protein coupled signal transduction pathway in response to PK1. A PK1 receptor also can bind to PK2 to induce PK1 receptor signaling. The amino acid sequence referenced as SEQ ID NO:7, which corresponds to the originally recognized human PK1 receptor, is encoded by the nucleotide sequence referenced as SEQ ID NO:10. A short isoform of human PK2 receptor is referenced as SEQ ID NO:6, which is encoded by SEQ ID NO:10 beginning at nucleotide 25.

The invention provides a short isoform of PKR2 (SEQ ID NO:17). This short isoform polypeptide has been localized to the hypothalamus and thalamus, which are crucial circadian clock targets.

The amino acid sequences of invention short and long and short variant PK receptor isoforms, although different at their N-termini, can be substantially identical to originally recognized PK1 and PK2 receptor isoforms in the remaining amino acid sequence; or can contain minor modifications in the remaining amino acid sequence with respect to the originally recognized PK1 and PK2 receptor isoforms, so long as PK receptor activity remains substantially preserved.

Such a minor modification of a PK1 or PK2 receptor isoform or splice variant can be, for example, a substitution, deletion or addition of one or more amino acids. Thus, minor modification of the sequence referenced as SEQ ID NO:2, 3, 4, 5, 6 or 17 can have one or more additions, deletions, or substitutions of natural or non-natural amino acids relative to the native polypeptide sequence. Such a modification can be, for example, a conservative change, wherein a substituted amino acid has similar structural or chemical properties, for example, substitution of an apolar amino acid with another apolar amino acid (such as replacement of leucine with isoleucine). Such a modification can also be a nonconservative change, wherein a substituted amino acid has different but sufficiently similar structural or chemical properties so as to not adversely affect the desired biological activity, such as, replacement of an amino acid with an uncharged polar R group with an amino acid with an apolar R group (such as replacement of glycine with tryptophan). Further, a minor modification of a human PK2 or PK1 receptor isoform amino acid sequence referenced as SEQ ID NO:2, 3, 4, 5, 6 or 17 can be the substitution of an L-configuration amino acid with the corresponding D-configuration amino acid with a non-natural amino acid.

In addition, a minor modification can be a chemical or enzymatic modification to the polypeptide, such as replacement of hydrogen by an alkyl, acyl, or amino group; esterification of a carboxyl group with a suitable alkyl or aryl moiety; alkylation of a hydroxyl group to form an ether derivative; phosphorylation or dephosphorylation of a serine, threonine or tyrosine residue; or N- or O-linked glycosylation.

Those skilled in the art can determine whether minor modifications to the sequence of a PK1 or PK2 receptor variant isoform are desirable. Such modifications can be made, for example, to enhance the stability, bioavailability or bioactivity of the receptor. A modified PK1 or PK2 receptor variant isoform polypeptide can be prepared, for example, by recombinant methods, by synthetic methods, by post-synthesis chemical or enzymatic methods, or by a combination of these methods, and tested for ability to bind PK2 or PK1 or signal through a G-protein coupled signal transduction pathway.

Those skilled in the art also can determine regions in a PK1 or PK2 receptor amino acid sequence that can be modified without abolishing ligand binding or signaling through a G-protein coupled signal transduction pathway. Structural and sequence information can be used to determine the amino acid residues important for PK2 receptor or PK1 receptor activity. For example, comparisons of amino acid sequences of PK2 receptor or PK1 receptor sequences from different species can provide guidance in determining amino acid residues that can be altered without abolishing activity. Further, a large number of published GPCR structure-function studies have indicated regions of GPCRs involved in ligand interaction, G-protein coupling and in forming transmembrane regions, and indicate regions of GPCRs tolerant to modification (see, for example, Burstein et al., J. Biol. Chem., 273(38): 24322-7 (1998) and Burstein et al., Biochemistry, 37(12): 4052-8 (1998)). In addition, computer programs known in the art can be used to determine which amino acid residues of a GPCR can be modified as described above without abolishing activity (see, for example, Eroshkin et al., Comput. Appl. Biosci. 9:491-497 (1993)).

The invention provides an isolated nucleic acid molecule comprising a sequence that encodes a variant isoform of human prokineticin receptor 2 (PRK2), wherein the isoform has an amino acid sequence selected from SEQ ID NOS:2, 3, and 4, and optionally can contain a heterologous sequence, such as a tag. An exemplary nucleic acid molecule of the invention has substantially the same nucleotide sequence as SEQ ID NO:8, or a fragment thereof. The invention also provides an isolated nucleic acid molecule comprising a sequence that encodes a short isoform of human prokineticin receptor 2 (PRK2), wherein the isoform has amino acid sequence SEQ ID NO:5 and optionally can contain a heterologous sequence, such as a tag.

A nucleic acid molecule of the invention can be linked to a variety of heterologous nucleotide sequences, which can be, for example, a nucleic acid encoding a tag. Such a tag can be, for example, a purification tag useful in the isolation of the encoded polypeptide, or a detection tag.

The invention further provides an isolated nucleic acid molecule comprising a sequence that encodes a short isoform of human prokineticin receptor 1 (PKR1), wherein the isoform has the amino acid sequence referenced as SEQ ID NO:6, and optionally can contain a heterologous sequence, such as a tag.

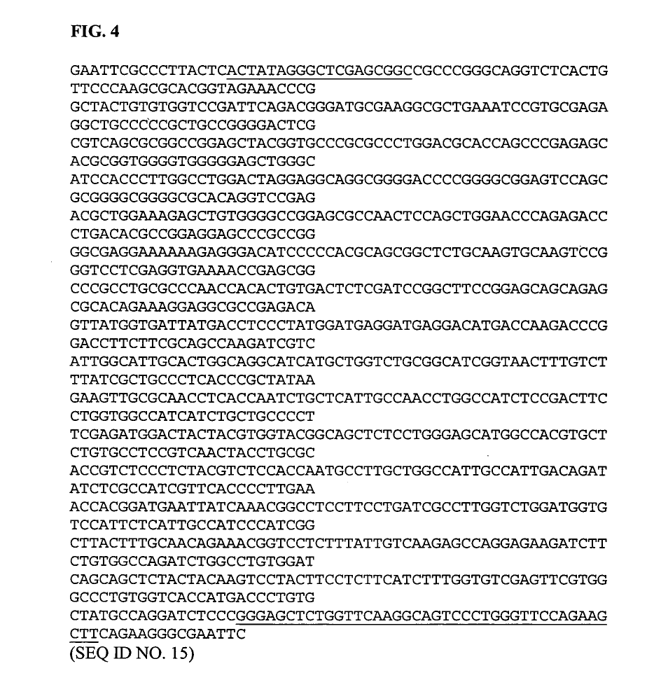

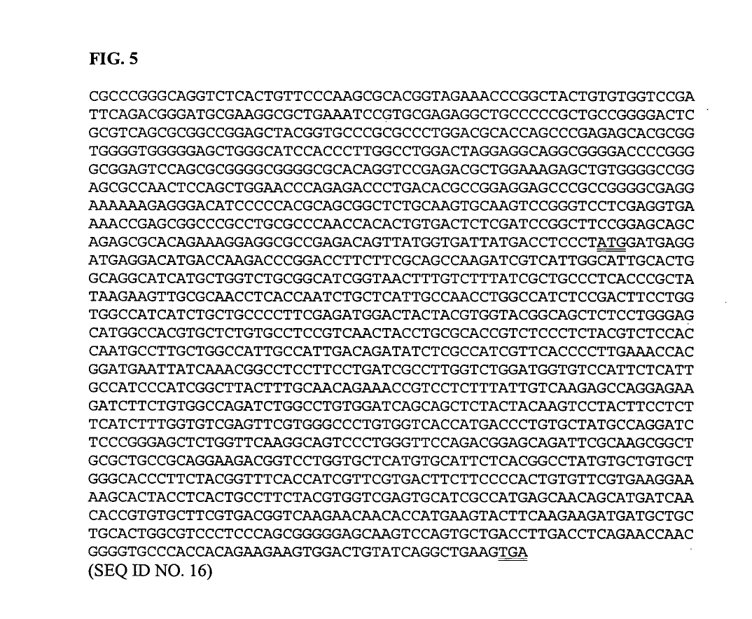

The invention further provides an isolated nucleic acid molecule comprising a sequence that encodes for a short isoform of human prokineticin receptor 2 (PKR2), wherein the short isoform has the amino acid sequence referenced as SEQ ID NO:17, and optionally can contain a heterologous sequence, such as a tag. An example of such a nucleic acid comprises the nucleic acid sequence shown in FIG. 5 as SEQ ID NO:16.

Further provided by the invention are vectors that contain a nucleic acid molecule of the invention, and isolated host cells containing the plasmid. Exemplary vectors include vectors derived from a virus, such as a bacteriophage, a baculovirus or a retrovirus, and vectors derived from bacteria or a combination of bacterial sequences and sequences from other organisms, such as a cosmid or a plasmid. The vectors of the invention will generally contain elements such as an origin of replication compatible with the intended host cells; transcription termination and RNA processing signals; one or more selectable markers compatible with the intended host cells; and one or more multiple cloning sites. Optionally, the vector will further contain sequences encoding tag sequences, such as GST tags, and/or a protease cleavage site, such as a Factor Xa site, which facilitate expression and purification of the encoded polypeptide.

The choice of particular elements to include in a vector will depend on factors such as the intended host cells; the insert size; whether expression of the inserted sequence is desired; the desired copy number of the vector; the desired selection system, and the like. The factors involved in ensuring compatibility between a host cell and a vector for different applications are well known in the art.

In applications in which the vectors are to be used for recombinant expression of the encoded polypeptide, the isolated nucleic acid molecules will generally be operatively linked to a promoter of gene expression, which may be present in the vector or in the inserted nucleic acid molecule. An isolated nucleic acid molecule encoding a PK receptor isoform can be operatively linked to a promoter of gene expression. As used herein, the term “operatively linked” means that the nucleic acid molecule is positioned with respect to either the endogenous promoter, or a heterologous promoter, in such a manner that the promoter will direct the transcription of RNA using the nucleic acid molecule as a template.

Methods for operatively linking a nucleic acid to a heterologous promoter are well known in the art and include, for example, cloning the nucleic acid into a vector containing the desired promoter, or appending the promoter to a nucleic acid sequence using PCR. A nucleic acid molecule operatively linked to a promoter of RNA transcription can be used to express prokineticin transcripts and polypeptides in a desired host cell or in vitro transcription-translation system.

The choice of promoter to operatively link to an invention nucleic acid molecule will depend on the intended application, and can be determined by those skilled in the art. For example, if a particular gene product may be detrimental to a particular host cell, it may be desirable to link the invention nucleic acid molecule to a regulated promoter, such that gene expression can be turned on or off. Alternatively, it may be desirable to have expression driven by either a weak or strong constitutive promoter. Exemplary promoters suitable for mammalian cell systems include, for example, the SV40 early promoter, the cytomegalovirus (CMV) promoter, the mouse mammary tumor virus (MMTV) steroid-inducible promoter, and the Moloney murine leukemia virus (MMLV) promoter. Exemplary promoters suitable for bacterial cell systems include, for example, T7, T3, SP6, lac and trp promoters. An exemplary vector suitable for fusion protein expression in bacterial cells is the pGEX-3X vector (Amersham Pharmacia Biotech, Piscataway, N.J.).

Also provided are cells containing an isolated nucleic acid molecule encoding a short or long PK receptor isoform. The isolated nucleic acid molecule will generally be contained within a vector, and can be maintained episomally, or incorporated into the host cell genome.

The cells of the invention can be used, for example, for molecular biology applications such as expansion, subcloning or modification of the isolated nucleic acid molecule. For such applications, bacterial cells, such as laboratory strains of E. coli, are useful, and expression of the encoded polypeptide is not required.

The cells of the invention can also be used to recombinantly express and isolate the encoded polypeptide. For such applications bacterial cells (e.g. E. coli), insect cells (e.g. Drosophila), yeast cells (e.g. S. cerevisiae, S. pombe, or Pichia pastoris), and vertebrate cells (e.g. mammalian primary cells and established cell lines; and amphibian cells, such as Xenopus embryos and oocytes). An exemplary cell suitable for recombinantly expressing prokineticin polypeptides is an E. coli BL21 cell.

The invention also provides methods for preparing an isolated polypeptide corresponding to a short or long isoform PKR, by culturing host cells so as to express a recombinant prokineticin polypeptide. A variety of well-known methods can be used to introduce a vector into a host cell for expression of a recombinant polypeptide (see, for example, Sambrook et al., Molecular Cloning: A Laboratory Manual, Cold Spring Harbor Laboratory, New York (1992) and Ansubel et al., Current Protocols in Molecular Biology, John Wiley and Sons, Baltimore, Md. (1998)). The selected method will depend, for example, on the selected host cells.

An isolated polypeptide of the invention can be prepared by biochemical procedures, and can be isolated from host cells that recombinantly express the polypeptide, or from tissues or cells that normally express the polypeptides. A variety of well-known biochemical procedures routinely used in the art, including membrane fractionation, chromatography, electrophoresis and ligand affinity methods, and immunoaffinity methods with the prokineticin antibodies described herein, can be used. An isolated polypeptide of the invention can also be prepared by chemical synthesis procedures known in the art. Following chemical synthesis, an inactive prokineticin can be refolded by the methods described herein to restore activity.

If desired, such as to optimize their functional activity, selectivity, stability or bioavailability, chemically synthesized polypeptides can be modified to include D-stereoisomers, non-naturally occurring amino acids, and amino acid analogs and mimetics. Examples of modified amino acids and their uses are presented in Sawyer, Peptide Based Drug Design, ACS, Washington (1995) and Gross and Meienhofer, The Peptides: Analysis, Synthesis, Biology, Academic Press, Inc., New York (1983). For certain applications, it can also be useful to incorporate one or more detectably labeled amino acids into a chemically synthesized polypeptide or peptide, such as radiolabeled or fluorescently labeled amino acids.

As used herein, the term “isolated” indicates that the molecule is altered by the hand of man from how it is found in its natural environment. An “isolated” prokineticin polypeptide can be a “substantially purified” molecule, that is at least 60%, 70%, 80%, 90 or 95% free from cellular components with which it is naturally associated. An isolated polypeptide can be in any form, such as in a buffered solution, a suspension, a lyophilized powder, recombinantly expressed in a heterologous cell, bound to a receptor or attached to a solid support.

The invention also provides an antibody selective for a short or long isoform of PKR2, such as those referenced as SEQ ID NOS:2-5 and 17; and a short isoform of PKR1, such as that referenced as SEQ ID NO:6. An antibody that selectively binds to a short isoform of native PKR2 can bind to amino acid sequence SEQ ID NO:5, without substantially binding to amino acid sequence SEQ ID NO:1. Such antibodies can bind selectively to a native, or non-denatured, short isoform of a PK receptor without substantially binding to a native originally recognized isoform of a PK receptor when, for example, the native short isoform has a different conformation than the corresponding native longer isoform.

An antibody that selectively binds to a long isoform of PKR2 can bind, for example, to SEQ ID NO:2, 3, or 4, without substantially binding to SEQ ID NO:1. Such an antibody can bind selectively to a long isoform of a PK receptor without substantially binding to a shorter isoform, such as an originally recognized isoform of a PK receptor, because the long isoform contains amino acids not found in shorter isoforms.

An antibody that selectively binds to a short isoform of PKR2 can bind, for example, to SEQ ID NO:17 without substantially binding to SEQ ID NO:1. Such an antibody can bind selectively to a short isoform PKR2 without substantially binding to one of the other isoforms, such as the originally recognized isoform of the PK receptor, because the short PKR2 isoform contains amino acids not found in the other isoforms.

The antibodies of the invention can be used, for example, to detect expression of a short or long isoform of a PK receptor in research and diagnostic applications. Such antibodies are also useful for identifying nucleic acid molecules that encode a short or long isoform of a PK receptor present in mammalian expression libraries, and for purifying PK receptor polypeptides by immunoaffinity methods. Furthermore, such antibodies can be administered therapeutically to bind to and block the activity of an isoform of a PK receptor, such as in applications in which it is desirable to modulate, for example, GI smooth muscle contraction or motility; circadian rhythm function; angiogenesis; or gastric acid or pepsinogen secretion.

The term “antibody,” as used herein, is intended to include molecules having selective binding activity for an amino acid sequence corresponding to a short or long isoform of a PK receptor of at least about 1×10⁵ M⁻¹, preferably at least 1×10⁷ M⁻¹, more preferably at least 1×10⁹ M⁻¹. The term “antibody” includes both polyclonal and monoclonal antibodies, as well as antigen binding fragments of such antibodies (e.g. Fab, F(ab′)₂, Fd and Fv fragments and the like). In addition, the term “antibody” is intended to encompass non-naturally occurring antibodies, including, for example, single chain antibodies, chimeric antibodies, bifunctional antibodies, CDR-grafted antibodies and humanized antibodies, as well as antigen-binding fragments thereof.

Methods of preparing and isolating antibodies, including polyclonal and monoclonal antibodies, using peptide and polypeptide immunogens, are well known in the art and are described, for example, in Harlow and Lane, Antibodies: A Laboratory Manual, Cold Spring Harbor Laboratory Press (1988). Non-naturally occurring antibodies can be constructed using solid phase peptide synthesis, can be produced recombinantly or can be obtained, for example, by screening combinatorial libraries consisting of variable heavy chains and variable light chains. Such methods are described, for example, in Huse et al. Science 246: 1275-1281 (1989); Winter and Harris, Immunol. Today 14: 243-246 (1993); Ward et al., Nature 341: 544-546 (1989); Hilyard et al., Protein Engineering: A practical approach (IRL Press 1992); and Borrabeck, Antibody Engineering, 2d ed. (Oxford University Press 1995).

The invention provides screening assays for identifying compounds that modulate PK receptor activity, such as agonists and antagonists of PK receptors. The agonists and antagonists identified using the methods of the invention can be used to beneficially modulate PK receptor activity to treat an individual having a condition associated with aberrant low or high level of PK receptor activity. For example, because PK2 receptors can mediate circadian rhythm function in animals (Cheng et al. Nature 247: 405-410 (2002)), a PK2 receptor modulating compound can be used to treat disorders of circadian rhythm function, such as sleep disorders, shift work disorders and seasonal depression. Similarly, because PK2 receptors can mediate angiogenesis in a variety of tissues (LeCouter et al., Nature 412: 877-884 (2001); Lin et al. J. Biol. Chem. 277: 19 (2002)), including endothelium, a PK2 receptor antagonist can be used to reduce or inhibit angiogenesis in PK receptor expressing tissues. Such an antagonist can be useful for treating ischemic heart disease, critical limb ischemia, wound healing and burns, cancer, diabetic retinopathy, inflammatory diseases such as arthritis and psoriasis, and female reproductive disorders such as menorrhagia, endometriosis, dysfunctional uterine bleeding, fibroids and adenoyosis. Also, because PK2 receptors can mediate gastric contractility and motility, as well as mediate secretion of gastric acid and pepsinogen, a PK2 receptor modulating drug can be used to increase or decrease production of gastric acid or pepsinogen to treat, for example, gastric reflux disorder (GERD) irritable bowel syndrome, postoperative ileus, diabetic gastroparesis, chronic constipation and reducing side effects of chemotherapy. Finally, because PK2 receptors can mediate neurogenesis, a PK receptor modulating drug can be used to treat neurological disorders.

A short or long isoform of a PK receptor of the invention can be used in a variety of screening assays for identifying an antagonist or agonist of a PK receptor. For example, a selected a short or long isoform of a PK receptor can be used to identify an antagonist or agonist of the selected isoform, or two or more PKR2 isoforms, including the originally recognized PKR2 isoform. Likewise, a short PKR1 isoform can be used to identify an antagonist or agonist of the short isoform, or both the short isoform and the originally recognized PKR2 isoform.

As used herein, the term “prokineticin receptor antagonist” means a compound that selectively inhibits or decreases normal signal transduction through a PK receptor, which can be any isoform of a PK receptor. A PK receptor antagonist can act by any antagonistic mechanism, such as by binding a PK receptor or PK, thereby inhibiting binding between PK and PK receptor. A PK receptor antagonist can also inhibit binding between a specific or non-specific PK receptor agonist and PK receptor. Such a specific or non-specific PK receptor agonist can be, for example, a drug that produces unwanted side effects by promoting signaling through the PK receptor. A PK receptor antagonist can also act, for example, by inhibiting the binding activity of PK or signaling activity of PK receptor. For example, a PK receptor antagonist can act by altering the state of phosphorylation or glycosylation of PK receptor. A PK receptor antagonist can also be an inverse agonist, which decreases PK receptor signaling from a baseline amount of constitutive PK receptor signaling activity.

As used herein, the term “prokineticin receptor agonist” means a compound that selectively promotes or enhances normal signal transduction through a prokineticin receptor, which can be any isoform of the PK receptor. A PK receptor agonist can act by any agonistic mechanism, such as by binding a prokineticin receptor at the normal prokineticin (PK) binding site, thereby promoting PK receptor signaling. A PK receptor agonist can also act, for example, by potentiating the binding activity of PK or signaling activity of PK receptor. An agonist of a PK2 receptor also can function as an agonist of a PK1 receptor because PK1 and PK2 both can bind to PKR1 and PKR2. As such, a PK1 receptor agonist can be tested for its ability to function as a PK2 receptor agonist using the screening methods described herein; and a PK2 receptor agonist can be tested for its ability to function as a PK1 receptor agonist using the screening methods described herein.

Specific examples of PK receptor agonists include the human and mouse PK2 and PK1 amino acid sequences shown in FIG. 2, as well as the toad Bv8 amino acid sequence; frog Bv8 amino acid sequence, snake MIT1 amino acid sequence, and chimeric PK1-PK2 amino acid sequences also shown in FIG. 2.

A screening assay used in a method of the invention for identifying a PK receptor agonist or antagonist can involve detecting a predetermined signal produced by a PK receptor. As used herein, the term “predetermined signal” is intended to mean a readout, detectable by any analytical means, that is a qualitative or quantitative indication of activation of G-protein-dependent signal transduction through PK2 receptor. Assays used to determine such qualitative or quantitative activation of G-protein-dependent signal transduction through PK2 receptor, are referred to below as “signaling assays.” G-proteins, or heterotrimeric GTP binding proteins, are signal transducing polypeptides having subunits designated Gα, Gβ and Gγ, that couple to seven-transmembrane cell surface receptors. G-proteins couple to such receptors to transduce a variety of extracellular stimuli, including light, neurotransmitters, hormones and odorants to various intracellular effector proteins. G-proteins are present in both eukaryotic and prokaryotic organisms, including mammals, other vertebrates, flies and yeast.

A signaling assay can be performed to determine whether a candidate compound is a PK receptor agonist or antagonist. In such an assay, a PK receptor, such as a short or long PKR2 isoform or a short PKR1 isoform, is contacted with one or more candidate compounds under conditions wherein the PK receptor produces a predetermined signal in response to a PK agonist, such as PK1 or PK2. In response to PK receptor activation, a predetermined signal can increase or a decrease from an unstimulated PK receptor baseline signal. A predetermined signal is an increasing signal, for example, when the amount of detected second messenger molecule is increased in response to PK receptor activation. A predetermined signal is a decreasing signal, for example, when the detected second messenger molecule is destroyed, for example, by hydrolysis, in response to PK receptor activation. A predetermined signal in response PK receptor activation can therefore be an increase in a predetermined signal that correlates with increased PK receptor activity, or a decrease in a predetermined signal that correlates with increased PK receptor activity. Accordingly, a PK receptor signaling assay of can be used to identify a PK receptor agonist that promotes production of a predetermined signal, whether the agonist promotes an increase in a predetermined signal that positively correlates with PK receptor activity, or a decrease in a predetermined signal that negatively correlates with PK receptor activity. Similarly, a signaling assay can be performed to determine whether a candidate compound is a PK receptor antagonist. In such a signaling assay, a PK receptor is contacted with one or more candidate compounds under conditions wherein the PK receptor produces a predetermined signal in response to a PK receptor agonist, such as PK, and a compound is identified that reduces production of the predetermined signal.

Signaling through G proteins can lead to increased or decreased production or liberation of second messengers, including, for example, arachidonic acid, acetylcholine, diacylglycerol, cGMP, cAMP, inositol phosphate, such as inositol-1,4,5-trisphosphate, and ions, including Ca⁺⁺ ions; altered cell membrane potential; GTP hydrolysis; influx or efflux of amino acids; increased or decreased phosphorylation of intracellular proteins; or activation of transcription.

Various assays, including high throughput automated screening assays, to identify alterations in G-protein coupled signal transduction pathways are well known in the art. Various screening assay that measure Ca⁺⁺, cAMP, voltage changes and gene expression are reviewed, for example, in Gonzalez et al., Curr. Opin. in Biotech. 9: 624-631 (1998); Jayawickreme et al., Curr. Opin. Biotech. 8: 629-634 (1997); and Coward et al., Anal. Biochem. 270: 2424-248 (1999). Yeast cell-based bioassays for high-throughput screening of drug targets for G-protein coupled receptors are described, for example, in Pausch, Trends in Biotech. 15: 487-494 (1997). A variety of cell-based expression systems, including bacterial, yeast, baculovirus/insect systems and mammalian cells, useful for detecting G-protein coupled receptor agonists and antagonists are reviewed, for example, in Tate et al., Trends in Biotech. 14: 426-430 (1996).

Assays to detect and measure G-protein-coupled signal transduction can involve first contacting a sample containing an isoform of a PKR1 or PKR2, such as an isolated cell, membrane or artificial membrane, such as a liposome or micelle, with a detectable indicator. A detectable indicator can be any molecule that exhibits a detectable difference in a physical or chemical property in the presence of the substance being measured, such as a color change. Calcium indicators, pH indicators, and metal ion indicators, and assays for using these indicators to detect and measure selected signal transduction pathways are described, for example, in Haugland, Molecular Probes Handbook of Fluorescent Probes and Research Chemicals, Sets 20-23 and 25 (1992-94). For example, calcium indicators and their use are well known in the art, and include compounds like Fluo-3 AM, Fura-2, Indo-1, FURA RED, CALCIUM GREEN, CALCIUM ORANGE, CALCIUM CRIMSON, BTC, OREGON GREEN BAPTA, which are available from Molecular Probes, Inc., Eugene Oreg., and described, for example, in U.S. Pat. Nos. 5,453,517, 5,501,980 and 4,849,362.

If desired, a predetermined signal other than Ca²⁺ influx can be used as the readout for PK2 receptor activation. The specificity of a G-protein for cell-surface receptors is determined by the C-terminal five amino acids of the Gα subunit. The nucleotide sequences and signal transduction pathways of different classes and subclasses of Gα subunits in a variety of eukaryotic and prokaryotic organisms are well known in the art. Thus, any convenient G-protein mediated signal transduction pathway can be assayed by preparing a chimeric Gα containing the C-terminal residues of a Gα that couples to a novel isoform of a PK2 receptor or PK1 receptor, such as Gαq, with the remainder of the protein corresponding to a Gα that couples to the signal transduction pathway it is desired to assay. Methods of recombinantly expressing chimeric Gα proteins are known in the art and are described, for example, in Conklin et al., Nature 363: 274-276 (1993), Komatsuzaki et al., FEBS Letters 406: 165-170 (1995), and Saito et al., Nature 400: 265-269 (1999). Additionally, chimeric Gα proteins can be prepared by synthetic methods.

Another type of signaling assay involves determining changes in gene expression in response to a PK receptor agonist or antagonist. A variety of signal transduction pathways contribute to the regulation of transcription in animal cells by stimulating the interaction of transcription factors with genetic sequences termed response elements in the promoter regions of responsive genes. Assays for determining the interaction of transcription factors with promoter regions to stimulate gene expression are well known to those skilled in the art and are commercially available.

An assay to identify compounds that function as PK receptor agonists or antagonists are generally performed under conditions in which contacting the receptor with a known receptor agonist would produce a predetermined signal. If desired, the assay can be performed in the presence of a known PK receptor agonist, such as a PK1 or PK2. The agonist concentration can be within 10-fold of the EC₅₀. Thus, an agonist that competes with PK2, PK1 or a PK2/PK1 chimera, for signaling through the PK2 receptor, or indirectly potentiates the signaling activity of PK2, can be readily identified. Similarly, an agonist that competes with PK2, PK1 or a PK2/PK1 chimera for signaling through the PK1 receptor can be readily identified.

Likewise, an antagonist that prevents PK2, PK1 or a PK2/PK1 chimera from binding the PK2 receptor, or indirectly decreases the signaling activity of PK2 receptor, also can be identified. Similarly, an antagonist that prevents PK2, PK1 or a PK2/PK1 chimera from binding the PK1 receptor, or indirectly decreases the signaling activity of PK1 receptor, also can be identified. The candidate compound can be tested at a range of concentrations to establish the concentration where half-maximal signaling occurs; such a concentration is generally similar to the dissociation constant (Kd) for PK2 receptor binding.

A binding assay can be performed to identify compounds that are PK receptor agonists or antagonists. In such an assay, a novel isoform of a PK2 receptor or PK1 receptor can be contacted one or more candidate compounds under conditions in which PK binds to the selected receptor and a compound that binds to the selected receptor or that reduces binding of an agonist to selected receptor can be identified. Contemplated binding assays can involve detectably labeling a candidate compound, or competing an unlabeled candidate compound with a detectably labeled PK agonist, such as a PK2, PK1 or PK2/PK1 chimera. A detectable label can be, for example, a radioisotope, fluorochrome, ferromagnetic substance, or luminescent substance. Exemplary radiolabels useful for labeling compounds include ¹²⁵I, ¹⁴C and ³H. Methods of detectably labeling organic molecules, either by incorporating labeled amino acids into the compound during synthesis, or by derivatizing the compound after synthesis, are known in the art.

In order to determine whether a candidate compound decreases binding of detectably labeled novel isoform of a PK2 to PK2 receptor, the amount of binding of a given amount of the detectably labeled PK is determined in the absence of the candidate compound. Generally the amount of detectably labeled PK will be less than its K_{d}, for example, 1/10 of its K_{d}. Under the same conditions, the amount of binding of the detectably labeled PK2, PK1 or PK2/PK1 chimera in the presence of the candidate compound is determined. A decrease in binding due to a candidate compound characterized as a PK2 receptor ligand is evidenced by at least 2-fold less, such as at least 10-fold to at least 100-fold less, such as at least 1000-fold less, binding of detectably labeled PK2, PK1 or PK2/PK1 chimera to PK2 receptor in the presence of the candidate compound than in the absence of the candidate compound. An exemplary assay for determining binding of detectably labeled PK2, PK1 or PK2/PK1 chimera to PK2 receptor or PK1 receptor is the radioligand filter binding assay described in Li et al. Molecular Pharmacology 59: 692-698 (2001)).

Either low- and high-throughput assays suitable for detecting selective binding interactions between a receptor and a ligand include, for example, fluorescence correlation spectroscopy (FCS) and scintillation proximity assays (SPA) reviewed in Major, J. Receptor and Signal Transduction Res. 15: 595-607 (1995); and in Sterrer et al., J. Receptor and Signal Transduction Res. 17: 511-520 (1997)). Binding assays can be performed in any suitable assay format including, for example, cell preparations such as whole cells or membranes that contain PK2 receptor or PK1 receptor, or substantially purified PK2 receptor polypeptide or PK1 receptor, either in solution or bound to a solid support.

As used herein, the term “candidate compound” refers to any biological or chemical compound. For example, a candidate compound can be a naturally occurring macromolecule, such as a polypeptide, nucleic acid, carbohydrate, lipid, or any combination thereof. A candidate compound also can be a partially or completely synthetic derivative, analog or mimetic of such a macromolecule, or a small organic molecule prepared by combinatorial chemistry methods. If desired in a particular assay format, a candidate compound can be detectably labeled or attached to a solid support.

Methods for preparing large libraries of compounds, including simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2: 422-428 (1998); Tietze et al., Curr. Biol., 2: 363-371 (1998); Sofia, Mol. Divers. 3: 75-94 (1998); Eichler et al., Med. Res. Rev. 15: 481-496 (1995); and the like. Libraries containing large numbers of natural and synthetic compounds also can be obtained from commercial sources.

The number of different candidate compounds to test in the methods of the invention will depend on the application of the method. For example, one or a small number of candidate compounds can be advantageous in manual screening procedures, or when it is desired to compare efficacy among several predicted ligands, agonists or antagonists. However, it will be appreciated that the larger the number of candidate compounds, the greater the likelihood of identifying a compound having the desired activity in a screening assay. Additionally, large numbers of compounds can be processed in high-throughput automated screening assays.

Assay methods for identifying compounds that selectively bind to or modulate signaling through a PK2 receptor generally involve comparison to a control. One type of a “control” is a preparation that is treated identically to the test preparation, except the control is not exposed to the candidate compound. Another type of “control” is a preparation that is similar to the test preparation, except that the control preparation does not express the receptor, or has been modified so as not to respond selectively to PK2 or PK1. In this situation, the response of the test preparation to a candidate compound is compared to the response (or lack of response) of the control preparation to the same compound under substantially the same reaction conditions.

A compound identified to be an agonist or antagonist of one or more PK1 or PK2 receptor isoforms can be tested for its ability to modulate one or more effects on the function of a cell or animal. For example, a PK receptor agonist or antagonist can be tested for an ability to modulate circadian rhythm function, angiogenesis, gastrointestinal contraction and motility and secretion of gastric acid or pepsinogen, neurological conditions and pain.

Exemplary assays for determining for determining the effect of a compound on circadian rhythm function are described, for example, in Cheng et al. Nature 247: 405-410 (2002). Exemplary assays for determining the effect of a compound on angiogenesis are described, for example, in U.S. Pat. No. 5,753,230 and PCT publication WO 97/15666 and U.S. Pat. No. 5,639,725, which describe tumor model systems; Langer et al., Science 193: 707-72 (1976); O'Reilly, et al., Cell 79: 315-328 (1994); and U.S. Pat. No. 5,753,230. Exemplary assays for determining the effect of a compound on GI contraction and motility are described, for example, in Li et al. Mol Pharmacol. 59(4): 692-8 (2001), and Thomas et al., Biochem. Pharmacol. 51: 779-788 (1993).

Exemplary assays for determining for determining the effect of a compound on gastric acid or pepsinogen secretion are described, for example, in Soll, Am. J. Physiol 238:G366-G375 (1980); Sol and Walsh, Annu. Rev. Physiol. 41: 35-53(1979); Lavezzo et al., Int J Tissue React 6(2): 155-165 (1984)) and in isolated gastric mucosae (Rangachari, Am. J. Physiol. 236: E733-E737 (1979), Bunce et al. Br. J. Pharmacol 58: 149-156 (1976); and Lavezzo et al., Int J Tissue React 6(2): 155-165 (1984)); Howden et al., Aliment Pharmacol Ther 1(4): 305-315 (1987); Hirschowitz et al. J. Pharmacol Exp Ther 224(2): 341-5 (1983), and Wilson et al. Gig Dis Sci 29(9): 797-801 (1984).

Exemplary assays for determining the effect of a compound on neurological conditions include animal models of trauma due to stroke or neural injury are known in the art. One experimental model of stroke involves occluding the right middle cerebral artery and both common carotid arteries of rats for a short period, followed by reperfusion (Moore et al., J. Neurochem. 80: 111-118). An experimental model of CNS injury is the fluid percussion injury (FPI) model, in which moderate impact (1.5-2.0 atm) is applied to the parietal cerebral cortex (Akasu et al., Neurosci. Lett. 329: 305-308 (2002). Experimental models of spinal cord injury are also used in the art (Scheifer et al., Neurosci. Lett. 323: 117-120 (2002). Suitable models for neural damage due to oxidative stress, hypoxia, radiation and toxins are also known in the art.

Exemplary assays for determining the effect of a compound on pain include well-known animal models of pain, such as the Mouse Writhing Assay, the Tail Flick Assay, the Sciatic Nerve Ligation assay, the Formalin Test and the Dorsal Root Ganglia Ligation assay (see, for example, Bennett and Xie, Pain 33: 87-107 (1988); and Lee et al., Neurosci. Lett. 186: 111-114 (1995); Dewey et al., J. Pharm. Pharmacol. 21: 548-550 (1969); Koster et al., Fed. Proc. 18: 412 (1959); pain (Malmberg and Yaksh, The Journal of Pharmacology and Experimental Therapeutics 263: 136-146 (1992)).

Because isoforms of PK receptor can be correlated with disease, the presence of such isoforms can be used as a diagnostic or prognostication indicator. Analysis of PK receptor mRNA or polypeptide can be used in such diagnostic methods to identify the presence of an isoform of the PK receptor that correlates with a disease or condition. Direct sequencing, binding, or hybridization assays including PCR, RT-PCR, Northern blot, Southern blot, and RNAse protection can be used to detect a PK receptor isoform. For example, PCR amplification or RT-PCR amplification of a region of a known difference between the originally identified receptor (or particular isoform) and a diagnostic isoform disclosed herein, such as SEQ ID NOS:2, 3, 4, 5, or 6, can be used. Similarly, an antibody that binds to a region of known difference between the originally identified receptor (or particular isoform) and a diagnostic isoform can be used. Similarly, reverse transcription reactions coupled with PCR amplification can be used to identify a PK receptor isoform, such as SEQ ID NOS:2, 3, 4, 5, or 6. Any of these methods can be used to detect disease, monitor disease progression and/or regression, and to evaluate the effects of treatments based on the presence or absence of a PKR isoform.

It is understood that modifications which do not substantially affect the activity of the various embodiments of this invention are also included within the definition of the invention provided herein.

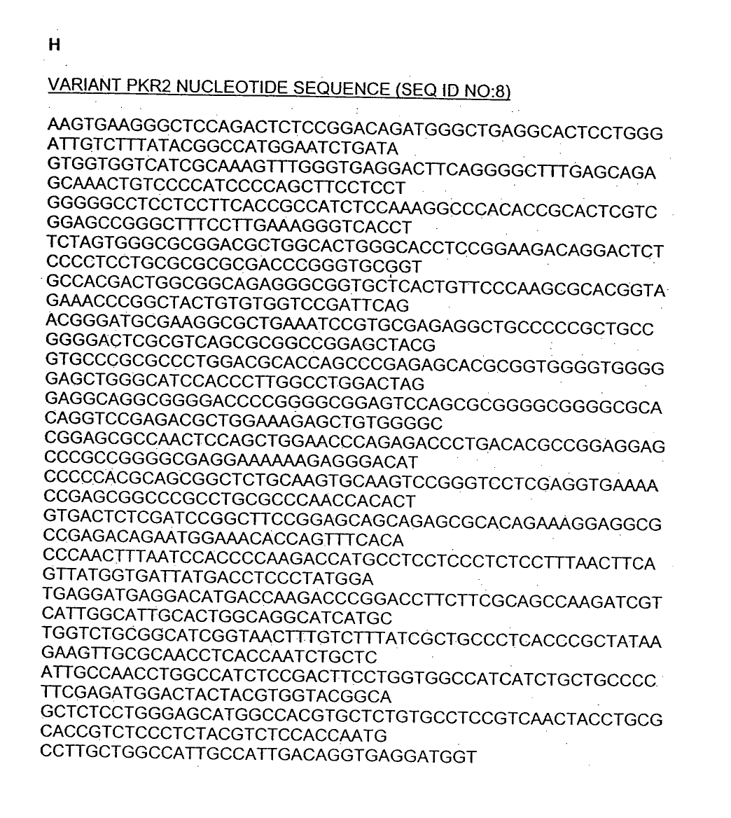

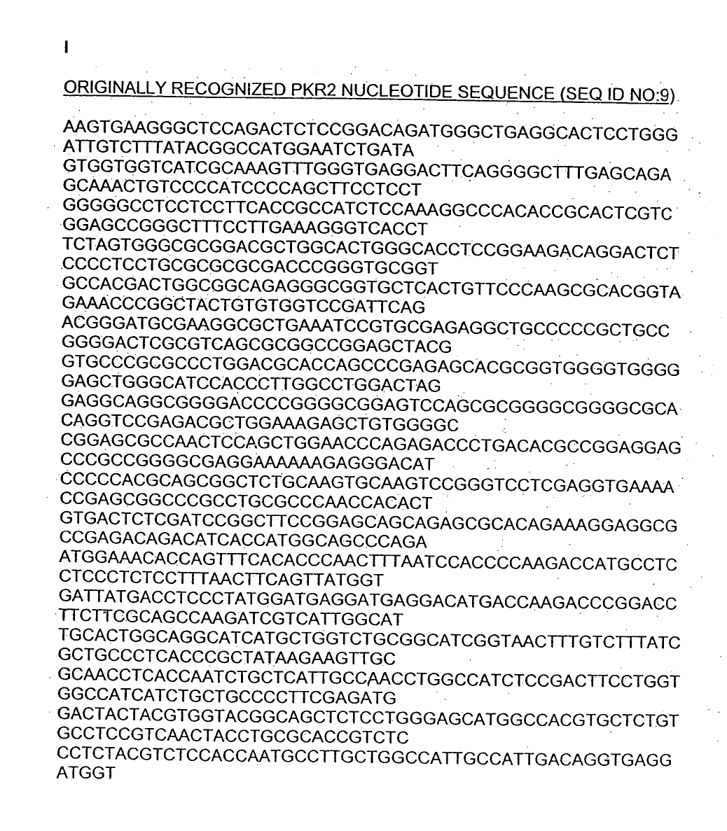

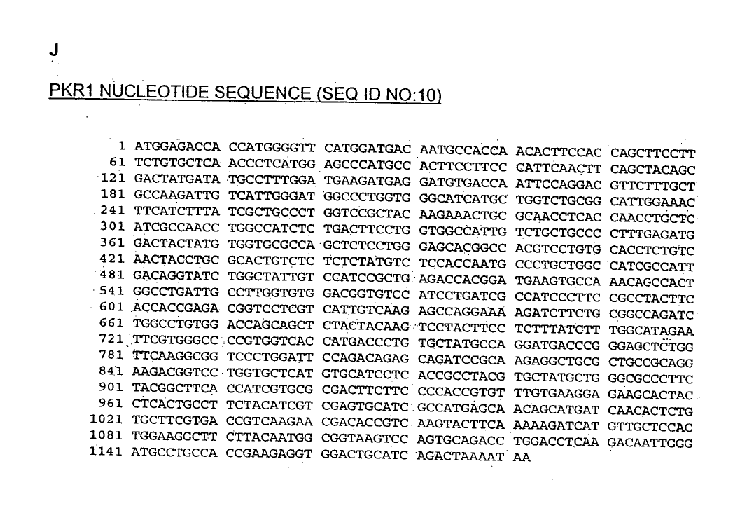

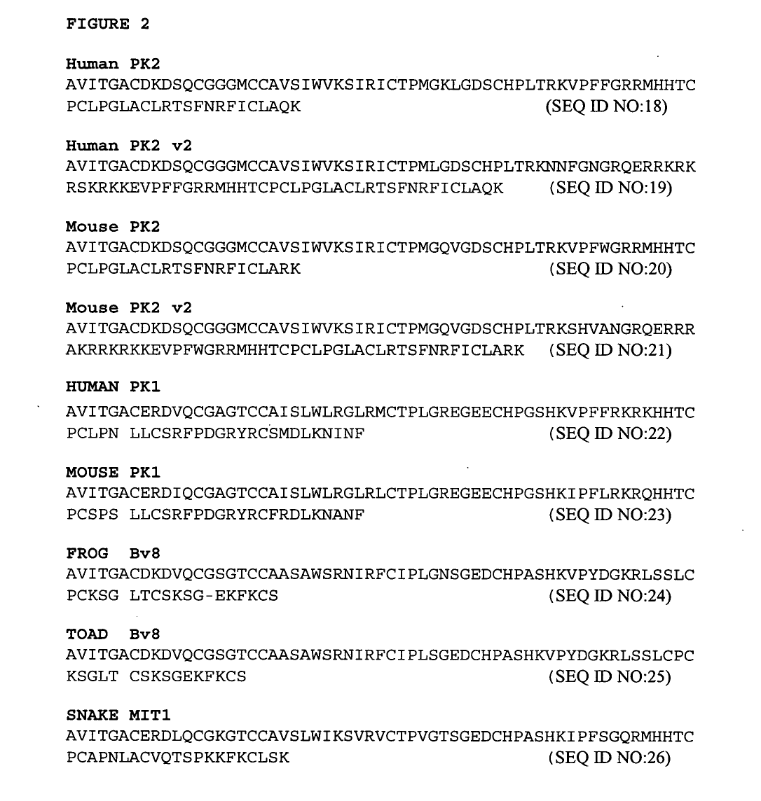

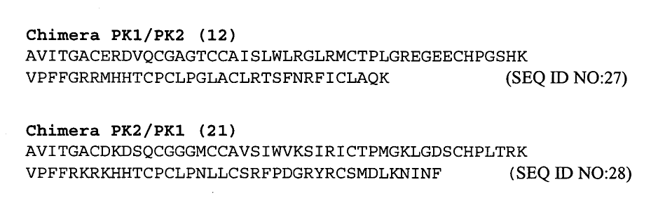

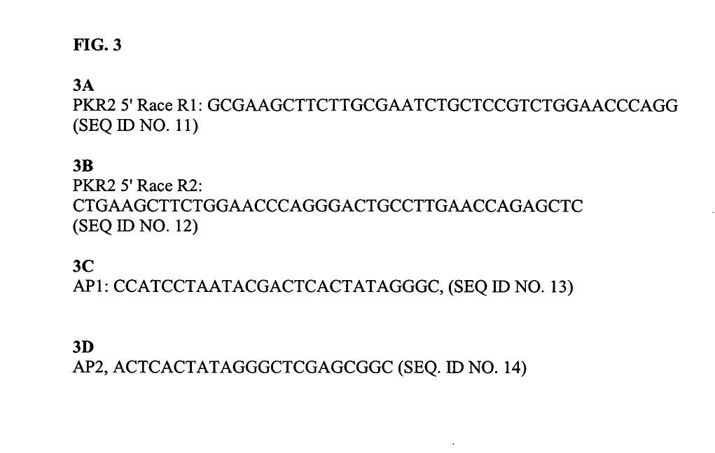

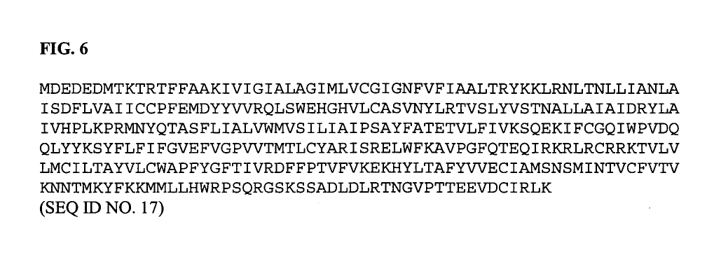

In order to identify splice variant isoforms of prokineticin receptors, one may use a variety of methods well known in the art. In some cases, a RACE protocol (Frohman, M. A., “RACE: Rapid Analysis of cDNA Ends,” In: PCR Protocols: A Guide to Methods and Applications Academic Press, N.Y. (1990)) may be employed to identify splice variants expressed in various cell lines, organs or tissues. In a particular example, human PKR2 mRNA was isolated from human hypothalamus and subjected to nested RACE using the primer set as follows: First PCR: 5′-RACE primer SEQ ID NO:11 (FIG. 3A); 3′-adaptor primer SEQ ID NO 13 (FIG. 3C). First PCR conditions: 94° C. for 30 minutes, followed by 30 cycles of 94° C. for 5 minutes and 68° C. for 4 minutes. Second PCR conditions: 30 cycles of 94° C. for 30 min and 72° C. for 2 min, each. 5′-RACE primer SEQ ID NO:12 (See FIG. 3B); 3′-adaptor primer SEQ ID NO:14 (See FIG. 3D). RACE was performed and the resulting PCR product (SEQ ID NO:15) isolated, subcloned into PCR2.1 (Invitrogen) and sequenced. The nucleotide sequence of human PKR2 isoform is shown in FIG. 5, SEQ ID NO:16. The isolated peptide has the sequence shown in FIG. 6, SEQ ID NO:17.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention.

1. An isolated prokineticin receptor 2 long isoform polypeptide, comprising an amino acid sequence selected from the amino acid sequences referenced as: SEQ ID NO:2, SEQ ID NO:3, SEQ ID NO:4 or SEQ ID NO:17.

2. An isolated prokineticin receptor 2 short isoform polypeptide, comprising the amino acid sequence referenced as SEQ ID NO:5.

3. An isolated prokineticin receptor 1 short isoform polypeptide, comprising the amino acid sequence referenced as SEQ ID NO:6.

4. An isolated prokineticin receptor 2 short isoform polypeptide, comprising the amino acid sequence referenced as SEQ ID NO:17.

5. A method for preparing an isolated polypeptide of claim 1 comprising culturing a host cell that expresses said polypeptide and substantially purifying the polypeptide.

6. A method for preparing an isolated polypeptide of claim 2 comprising culturing a host cell that expresses said polypeptide and substantially purifying the polypeptide.

7. A method for preparing an isolated polypeptide of claim 3 comprising culturing a host cell that expresses said polypeptide and substantially purifying the polypeptide.

8. A method for preparing an isolated polypeptide of claim 4 comprising culturing a host cell that expresses said polypeptide and substantially purifying the polypeptide.

9. An antibody that selectively binds the polypeptide of claim 1 without substantially binding to the amino acid sequence referenced as SEQ ID NO:1.

10. An antibody that selectively binds the polypeptide of claim 2 without substantially binding to the amino acid sequence referenced as SEQ ID NO:1.

11. An antibody that selectively binds the polypeptide of claim 3 without substantially binding to the amino acid sequence referenced as SEQ ID NO: 7.

12. An antibody that selectively binds the polypeptide of claim 4 without substantially binding to the amino acid sequence referenced as SEQ ID NO:1.

13. A method of identifying a prokineticin 2 receptor agonist, comprising contacting a preparation comprising a prokineticin 2 receptor isoform polypeptide selected from SEQ ID NO:2, SEQ ID NO:3, SEQ ID NO:4; SEQ ID NO:5 or SEQ ID NO:17, with one or more candidate compounds, and identifying a compound that selectively promotes production of a prokineticin 2 receptor signal, said compound being characterized as an agonist of said prokineticin 2 receptor isoform.

14. A method of identifying a prokineticin 2 receptor antagonist, comprising contacting a preparation comprising a prokineticin 2 receptor polypeptide isoform selected from SEQ ID NO:2, SEQ ID NO:3, SEQ ID NO:4, SEQ ID NO:5 or SEQ ID NO:17, with one or more candidate compounds in the presence of a prokineticin, and identifying a compound that selectively inhibits production of a prokineticin 2 receptor signal, said compound being characterized as an antagonist of said prokineticin 2 receptor isoform.

15. A method of identifying a prokineticin 1 receptor agonist, comprising contacting a preparation comprising a prokineticin 1 receptor polypeptide isoform referenced as SEQ ID NO:6, with one or more candidate compounds, and identifying a compound that selectively promotes production of a prokineticin 1 receptor signal, said compound being characterized as an agonist of said prokineticin 1 receptor isoform.

16. A method of identifying a prokineticin 1 receptor antagonist, comprising contacting a preparation comprising a prokineticin 1 receptor polypeptide isoform referenced as SEQ ID NO:6, with one or more candidate compounds in the presence of a prokineticin, and identifying a compound that selectively inhibits production of a prokineticin 1 receptor signal, said compound being characterized as an antagonist of said prokineticin 1 receptor isoform.
